(19) United States
(12) Plant Patent Application Publication
(10) Pub. No.: US 2014/0047610 P1
(43) Pub. Date: Feb. 13, 2014

(54) Heucherella plant named 'Buttered Rum'

(50) ;

(71) Applicant: Janet N. Egger, Wilsonville, OR (US)

(72) Inventor: Janet N. Egger, Wilsonville, OR (US)

(73) Assignee: Terra Nova Nurseries, Inc.

(21) Appl. No.: 13/507,935

(22) Filed: Aug. 7, 2012

(51) Int. Cl. A01H 5/00

(52) U.S. Cl. PLT/441

(57) ABSTRACT

A new and distinct Heucherella plant substantially as shown and described, characterized by spring leaves of orange amber with a strong red pattern along veins, summer leaves amber with red brown leaf centers, fall leaves red with darker leaf centers, distinctively lobed, palmately divided leaves, small white flowers on short gray purple flower stalks, a medium plant size with multiple crowns, and excellent vigor.

### BOTANICAL DENOMINATION

Heucherella

### VARIETY DESIGNATION

‘Buttered Rum’

### BACKGROUND OF THE INVENTION

The present invention relates to a new and distinct cultivar of Heucherella and given the cultivar name of ‘Buttered Rum’. Heucherella is in the family Saxifragaceae. Heucherella ‘Buttered Rum’ originated from a controlled cross between Heuchera K270-4 (a proprietary unreleased Heuchera hybrid), as the seed parent, and Tiarella 58-9 (a proprietary unreleased Tiarella hybrid), as the pollen parent.

Compared to the seed parent, Heuchera K270-4 (unpatented), the new cultivar has leaves that are deeply lobed and caramel to red brown colored with a red to dark blotch along the main veins compared to leaves that are shallowly lobed and tan with no blotch.

Compared to the pollen parent, Tiarella 58-9 (unpatented), the new cultivar has amber to red brown foliage rather than green.

Compared to Heucherella ‘Brass Lantern’ (U.S. Plant Pat. No. 22,396) and Heucherella ‘Sweet Tea’ (U.S. Plant Pat. No. 21,296), this new cultivar has a lower more compact habit, shorter flower spikes, and keeps the red tones in the foliage color all the time rather than only in the spring and fall.

This new Heucherella is distinguished by:

1. spring leaves of orange amber with a strong red pattern along veins;

2. summer leaves amber with red brown leaf centers,

3. fall leaves red with darker leaf centers.

4. distinctively lobed, palmately divided leaves,

5. small white flowers on short maroon flower stalks

6. medium plant size with multiple crowns, and

7. excellent vigor.

This new cultivar has been reproduced only by asexual propagation (division and tissue culture). Each of the progeny exhibits identical characteristics to the original plant. Asexual propagation by division and tissue culture using standard micropropagation techniques with terminal and lateral shoots, as done in Canby, Oreg., shows that the foregoing characteristics and distinctions come true to form and are established and transmitted through succeeding propagations. The present invention has not been evaluated under all possible environmental conditions. The phenotype may vary with variations in environment without a change in the genotype of the plant.

### BRIEF DESCRIPTION OF THE DRAWINGS

FIG. 1 shows a six-month-old Heucherella ‘Buttered Rum’ growing in a one gallon container in a cool greenhouse in early May Canby, Oreg.

FIG. 2 shows a one-year-old Heucherella ‘Buttered Rum’ in bloom, planted in the ground in a trial bed in the shade in late summer in Canby, Oreg.

FIG. 3 show a one-year-old grown in a one gallon container in the greenhouse in late July.

FIG. 4 show a 14-month-old plant in the trial bed in full sun in the winter.

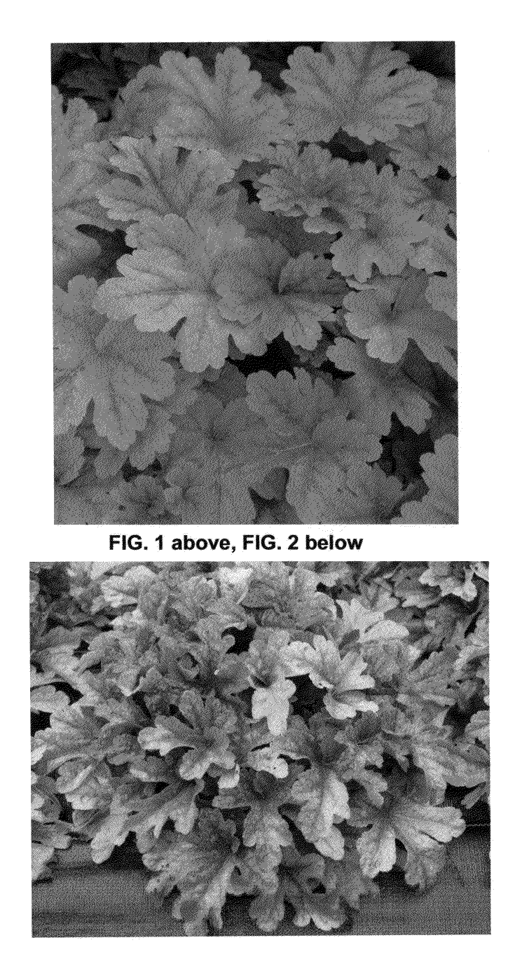

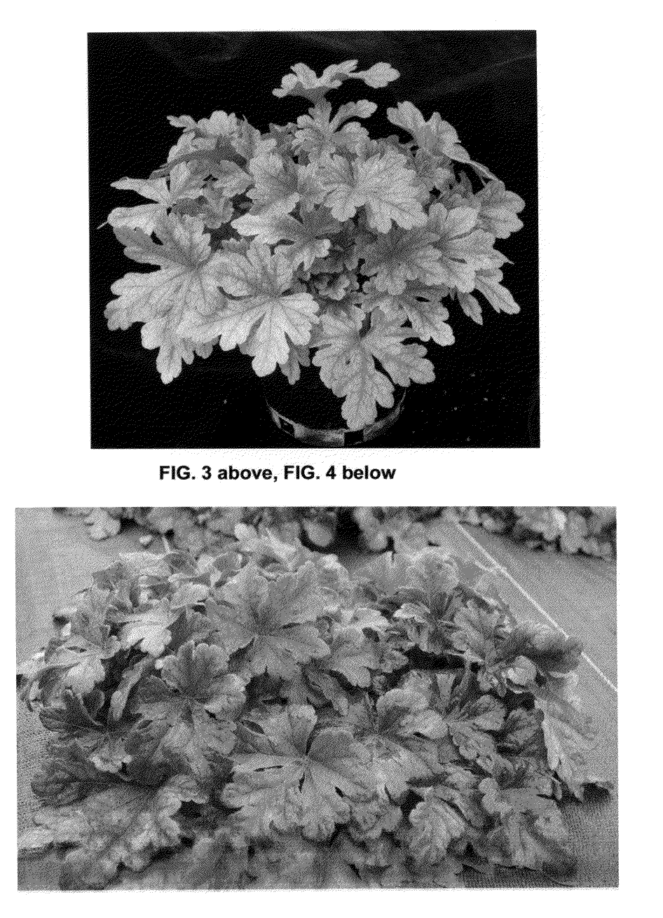

### DETAILED PLANT DESCRIPTION

The following is a detailed description of the new Heucherella cultivar based on observations of two-year-old specimens growing in one gallon containers in a cold greenhouse in Canby, Oreg. Canby is Zone 8 on the USDA Hardiness map. Temperatures range from a high of 95 degrees F. in August to an average of 32 degrees F. in January. Normal rainfall in Canby is 42.8 inches per year. The color descriptions are all based on The Royal Horticultural Society Colour Chart, fifth edition.
Plant:
   Form.—basal rosette, herbaceous perennial.
   Hardiness.—USDA Zone 4 to 9.
   Size.—19 cm tall from top of the foliage to the ground and 30 cm wide.
   Habit.—mounding.
   Vigor.—excellent.
   Roots.—fibrous, freely branching, fine, and white in color. Roots develop easily from cuttings.
Leaf:
   Type.—simple.
   Arrangement.—rosette.
   Shape.—ovate.
   Lobing/division.—5 lobes palmately parted with the terminal the longest and most deeply parted, each with distinctly cut secondary lobes (the depth of the lobes varies with plant maturity and leaf age; leaves become more deeply lobed with maturity).
   Venation.—palmate.
   Margins.—broadly crenate, older leaves undulate or twist slightly.
   Apex.—mucronulate.
   Base.—cordate.
   Blade size.—grows to 10.5 cm long and 8.5 cm wide.
   Surface texture.—glandular both surfaces.
   Petiole description.—grows to 13 cm long and 2 mm wide, glandular hairs, between Purple N77C and Greyed Purple 183D.
   Leaf color.—spring, topside, between Greyed Orange 174A and 65B with pattern Greyed Purple 183A, bottom side closest to Greyed Red 182B; summer topside newest leaves closest to Greyed Purple 183A (lightening to Greyed Orange 165C with the pattern Greyed Red 182A), bottom side closest to Greyed Purple 183C; fall Greyed Purple 185A with pattern Greyed Purple N186A, bottom side N79B; winter Greyed Purple 187B with pattern Greyed Purple N186A, bottom side N79B.
Inflorescence:
   Size.—grows to 2.3 cm wide and 10 cm long.
   Type.—raceme.
   Number of flowers per raceme.—about 60.
   Number of raceme.—24.
   Peduncle.—grows to 20 cm tall, 2 mm wide at base, Greyed Orange 166A, glandular hairs.
   Pedicel.—variable, with glandular hairs, Greyed Yellow 161A.
   Bloom period.—May with sporadic rebloom in Canby, Oreg.
Flower bud:
   Size.—2 mm wide and 3 mm long.
   Description.—glandular puberulent, ovoid.
   Color.—White NN155C.
Flower:
   Type.—imperfect.
   Shape.—campanulate.
   Size.—4 mm deep and 5 mm wide.
   Petal description.—5 in number, lanceolate, 4 mm long and 1 mm wide, some reflexed, tip acuminate, base attenuate, margin entire, glabrous on both sides, White NN155D on both sides.
   Calyx description.—4 mm deep and 2 mm wide, with 5 lobes, divided ¾ way to the base, campanulate, glandular hairs on outside, glabrous inside, tip obtuse, margin entire, NN155D on both sides.
   Stamen description.—none.
   Pistil description.—one central two beaked pistil, 4 mm long and 2 mm wide, White NN155D.
   Fragrance.—none.
   Lastingness.—each raceme blooms for about 2 weeks on the plant.
Fruit and seed: none
Disease and pest tolerance: The new cultivar is typical to the genus. No known resistances to pests or diseases. No problems have been found in Canby, Oreg.

I claim:

1. A new and distinct Heucherella plant as herein illustrated and described.
